(19) United States
(12) Patent Application Publication
(10) Pub. No.: US 2025/0186561 A1
(43) Pub. Date: Jun. 12, 2025

(54) BIOLOGICAL ENZYME FOR KILLING VIRUSES AND PREPARATION METHOD THEREOF

(71) Applicant: Changde Jizhi Biological Technology Co., Ltd, Changde (CN)

(72) Inventor: Xinqiao HE, Changde (CN)

(21) Appl. No.: 18/534,574

(22) Filed: Dec. 9, 2023

(51) Int. Cl. A61K 38/43; C12N 9/98

(52) U.S. Cl. CPC A61K 38/43; C12N 9/98

(57) ABSTRACT

A biological enzyme for killing viruses and a preparation method thereof are provided, which relate to the field of biotechnologies, and the biological enzyme is made from the following raw materials: Artemisia argyi, Lactuca sativa, Allium tuberosum, Allium cepiforme, Litsea rubescensLeeomte, Glycyrrhiza uralensis, Allium sativum, Mentha canadensis, Allium mongolicum, Lonicera japonica, Sargassum, monazite, calcium carbonate, fructo oligosaccharide (FOS), isomaltooligosaccharide (IMO) and organic selenium. The biological enzyme can be used for a H1N1 virus and its complications, and the biological enzyme does not harm beneficial fungi and cells of a human body, and makes up for deficiency that other antibiotics, drugs, disinfection and sterilization can not only kill bacteria and viruses, but also kill the beneficial fungi and cells.

### TECHNICAL FIELD

The disclosure relates to the field of biotechnologies, and more particularly to a biological enzyme for killing viruses and a preparation method thereof.

### BACKGROUND

Influenza viruses, pathogenic bacteria, and the like coexist with humans for a long time, and they threaten human health all the time. Preventing respiratory diseases such as influenza is still a long-term and uninterrupted major task for scientists to ensure hygiene and health. One of existing technical solutions for preventing viral infections is vaccination, which first helps prevent people from contracting the virus, but infection can still occur, especially when immunity of people begins to weaken. A main task of vaccines is to prevent severe illness or death. The vaccines require strict storage conditions, single administration routes, high costs, and are limited by vaccination time, making it difficult to solve various clinical symptoms and physical discomfort after infection.

At present, the main diseases that affect human health and life span are caused by bacteria, viruses and genes (typically cancer and leukemia). The bacteria have cell walls, can be independent of animals and plants, live in air and soil, and spread very widely. Various antibiotics invented by human beings can kill the bacteria without harming cells of human body, all antibiotics attack the cell walls and prevent cells of the bacteria from forming the cell walls during reproduction, thus the bacteria will naturally be killed. The viruses are exactly the same as human cells and have no cell walls. The viruses need to survive in animals, and once they leave the host's body, they will quickly die. In theory, the virus will not spread through air and objects, but it is not certain that the virus will not spread through air and objects, the virus will spread through droplets in a closed space, and will directly entry into the human body through the mouth, respiratory tract, eyes for infection.

### SUMMARY

A purpose of the disclosure is to provide a biological enzyme for killing viruses and a preparation method thereof, and the biological enzyme can be used for an influenza A (H1N1) and its complications. The biological enzyme of the disclosure does not harm beneficial fungi and cells of a human body, and makes up for deficiency that other antibiotics, drugs, disinfection and sterilization can not only kill bacteria and viruses, but also kill the beneficial fungi and cells.

Compared to the related art, the disclosure provides the following technical solutions.

A biological enzyme for killing viruses and a preparation method thereof are provided, and the biological enzyme is made from the following raw materials: 300-500 grams (g) of Artemisia argyi, 200-300 g of Lactuca sativa, 200-300 g of Allium tuberosum, 300-400 g of Allium cepiforme, 200-300 g of Litsea rubescensLeeomte, 50-150 g of Glycyrrhiza uralensis, 50-100 g of Allium sativum, 75-150 g of Mentha canadensis, 100-200 g of Allium mongolicum, 50-150 g of Lonicera japonica, 80-120 g of Sargassum, 20-30 g of monazite, 25-35 g of calcium carbonate, 10-20 g of fructo oligosaccharide (FOS), 8-12 g of isomaltooligosaccharide (IMO), and 20-40 g of organic selenium.

In an embodiment, the biological enzyme is made from the following raw materials: 300 g of the Artemisia argyi, 200 g of the Lactuca sativa, 200 g of the Allium tuberosum, 300 g of the Allium cepiforme, 200 g of the Litsea rubescensLeeomte, 50 g of the Glycyrrhiza uralensis, 50 g of the Allium sativum, 75 g of the Mentha canadensis, 100 g of the Allium mongolicum, 50 g of the Lonicera japonica, 80 g of the Sargassum, 20 g of the monazite, 25 g of the calcium carbonate, 10 g of the FOS, 8 g of the IMO, and 20 g of the organic selenium.

In an embodiment, the biological enzyme is made from the following raw materials: 500 g of the Artemisia argyi, 300 g of the Lactuca sativa, 300 g of the Allium tuberosum, 400 g of the Allium cepiforme, 300 g of the Litsea rubescensLeeomte, 150 g of the Glycyrrhiza uralensis, 100 g of the Allium sativum, 150 g of the Mentha canadensis, 200 g of the Allium mongolicum, 150 g of the Lonicera japonica, 120 g of the Sargassum, 30 g of the monazite, 35 g of the calcium carbonate, 20 g of the FOS, 12 g of the IMO, and 40 g of the organic selenium.

The preparation method of the biological enzyme for killing viruses includes:

(1) drying individually, according to the weight of the raw materials, the Lactuca sativa, the Allium tuberosum, the Litsea rubescensLeeomte, the Glycyrrhiza uralensis, the Allium mongolicum and the Lonicera japonica to obtain dried materials, placing the dried materials in a grinder for grinding to obtain grinded materials, decocting the grinded materials with purified water to obtain decocted materials, and filtering the decocted materials to obtain size (also referred to as thickened liquid);
merging the Artemisia argyi and the Mentha canadensis to obtain a mixture, soaking the mixture in water for 1 hour (h) to obtain a soaked mixture, distilling the soaked mixture by using a steam distillation to obtain a distilled mixture, extracting the distill mixture for once under reduced pressure by adding water to obtain a first extracted mixture, filtering the first extracted mixture to obtain a first filtrate, and concentrating the first filtrate to a first target relative density to obtain a first liquid medicine for later use; and
pulverizing the Allium sativum and the Allium cepiforme to obtain pulverized mixture, adding water to the pulverized mixture to perform a warm infusion in a closed environment for 3 h to obtain an infused mixture, adding ethanol into the infused mixture until an alcohol content of the infused mixture reaches 50% to obtain a mixture containing alcohol, extracting the mixture containing alcohol under reduced pressure to obtain a second extracted mixture, filtering the second extracted mixture to obtain a second filtrate, and concentrating the second filtrate to a second target relative density to obtain a second liquid medicine;

(2) adding the first liquid medicine, the second liquid medicine and the size into a mixing tank and stirring evenly to obtain a first mixed liquid;

(3) cooking the first mixed liquid obtained in step (2) after adding the Sargassum to obtain a cooked mixed liquid, filtering the cooked mixed liquid to obtain a filtered mixed liquid, and cooling the filtered mixed liquid to obtain a cooled mixed liquid;

(4) adding 20% of the weight of the FOS and 20% of the weight of the IMO into the cooled mixed liquid obtained in step (3) and stirring evenly to obtain an even mixed liquid, placing the even mixed liquid in a fermenter to obtain a first fermentation broth; filtering the first fermentation broth to obtain a filtered first fermentation broth, and placing the residual FOS, the residual IMO and the filtered first fermentation broth in the fermenter to obtain a second fermentation broth;

(5) pulverizing the monazite and the calcium carbonate to obtain powder; placing the powder in a furnace at a temperature of 2000-2200 degrees Celsius (° C.) to burn for 75-105 minutes (min) to obtain burned powder; cooling the burned powder to obtain cooled powder, placing the cooled powder in a cloth bag with 800-1200 mesh and then distilling the cooled powder with a steaming furnace to obtain a distillate for later use; and

(6) distilling the second fermentation broth to no distillate to obtain distilled fermentation broth for later use; completely mixing the distilled fermentation broth, the distillate obtained in step (5) and the organic selenium to obtain a second mixed liquid; and adding olive oil into the second mixed liquid with a volume ratio of the second mixed liquid and the olive oil of 1:5-10 to obtain the biological enzyme.

In an embodiment, an application method of the biological enzyme for killing viruses is provided, and the application method includes: applying the biological enzyme to prepare an antiviral product.

In an embodiment, the viruses include an influenza virus.

In an embodiment, the influenza virus is an influenza A virus.

In an embodiment, the influenza A virus is a H1N1 virus.

Beneficial effects of the disclosure are as follows.

The raw materials in the disclosure include the Artemisia argyi, the the Lactuca sativa, the Allium tuberosum, the Litsea rubescensLeeomte, the Glycyrrhiza uralensis, the Allium mongolicum and the Lonicera japonica, the Sargassum, the monazite, the calcium carbonate, the FOS, the IMO and the organic selenium. The above components contain various substances that are beneficial to a human body and can enhance immunity of the human body, and bacterial species used in fermentation are mixed to form a symbiotic colony, which can make the fermentation more complete. A product prepared in the disclosure contains various substances required by the human body, such as proteases, amino acids, polysaccharides, vitamins, enzymes, antioxidants, small molecule peptides and the like, which can improve the immunity of the human body, prevent diseases, inhibit cancer cells, has a good adjuvant therapeutic effect on cancer, and also has a good adjuvant therapeutic effect on diabetes, liver diseases, digestive system diseases and the like. In addition, through fermentation, macromolecular polysaccharides, polypeptides and other substances are decomposed into small molecular substances, for example, the polypeptides are decomposed into the small molecule peptides, which makes them more easily absorbed by the human body, meanwhile, mixed colonies can make the colonies in gastrointestinal system reach a good balance, eliminate harmful bacteria, and make the human body healthier.

### DETAILED DESCRIPTION OF EMBODIMENTS

In order to make those skilled in the art understand the disclosure better, technical solutions in embodiments of the disclosure will be clearly and completely described below. Apparently, the embodiments described are merely some of the embodiments of the disclosure, not all of them. According to the embodiments in the disclosure, all other embodiments obtained by those skilled in the art without creative work fall within a scope of protection of the disclosure.

### Embodiment 1

A biological enzyme for killing viruses and a preparation method thereof are provided, and the biological enzyme is made from the following raw materials: 300 grams (g) of Artemisia argyi, 200 g of Lactuca sativa, 200 g of Allium tuberosum, 300 g of Allium cepiforme, 200 g of Litsea rubescensLeeomte, 50 g of Glycyrrhiza uralensis, 50 g of Allium sativum, 75 g of Mentha canadensis, 100 g of Allium mongolicum, 50 g of Lonicera japonica, 80 g of Sargassum, 20 g of monazite, 25 g of calcium carbonate, 10 g of fructo oligosaccharide (FOS), 8 g of isomaltooligosaccharide (IMO), and 20 g of organic selenium.

The preparation method of the biological enzyme includes the following steps (1)-(6).

In step (1), the Lactuca sativa, the Allium tuberosum, the Litsea rubescensLecomte, the Glycyrrhiza uralensis, the Allium mongolicum and the Lonicera japonica are dried individually according to weight of the raw materials to obtain dried materials, the dried materials are placed in a grinder for grinding to obtain grinded materials, the grinded materials are decocted with purified water to obtain decocted materials, and the decocted materials are filtered to obtain size; and step (1) further includes the following steps 1 and 2.

In step 1, the Artemisia argyi and the Mentha canadensis are merged to obtain a mixture, the mixture is soaked in water for 1 hour (h) to obtain a soaked mixture, the soaked mixture is distilled by using a steam distillation to obtain a distilled mixture, the distilled mixture is extracted for once under reduced pressure by adding water to obtain a first extracted mixture, the first extracted mixture is filtered to obtain a first filtrate, and the first filtrate is concentrated to a first target relative density to obtain a first liquid medicine for later use.

In step 2, the Allium sativum and the Allium cepiforme are pulverized to obtain pulverized mixture, water is added to the pulverized mixture to perform a warm infusion in a closed environment for 3 h to obtain an infused mixture, ethanol is added into the infused mixture until an alcohol content reaches 50% to obtain a mixture containing alcohol, the mixture containing alcohol is extracted under reduced pressure to obtain a second extracted mixture, the second extracted mixture is filtered to obtain a second filtrate, and the second filtrate is concentrated to a second target relative density to obtain a second liquid medicine.

In step (2), the first liquid medicine, the second liquid medicine and the size are added into a mixing tank, after stirring evenly, a first mixed liquid is obtained.

In step (3), the first mixed liquid obtained in step (2) is cooked after adding the Sargassum to obtain a cooked mixed liquid, the cooked mixed liquid is filtered to obtain a filtered mixed liquid, and the filtered mixed liquid is cooled to obtain a cooled mixed liquid.

In step (4), 20% of the weight of the FOS and 20% of the weight of the IMO are added into the cooled mixed liquid obtained in step (3), after stirring evenly, an even mixed liquid is obtained, the even mixed liquid is placed in a fermenter to obtain a first fermentation broth; and the first fermentation broth is filtered to obtain a filtered first fermentation broth, and residual FOS, residual IMO and the filtered first fermentation broth are placed in the fermenter to obtain a second fermentation broth.

In step (5), the monazite and the calcium carbonate are pulverized to obtain powder; the powder is placed in a furnace at a temperature of 2000 degrees Celsius (° C.) to burn for 105 minutes (min) to obtain burned powder; the burned powder is cooled to obtain cooled powder, the cooled powder is placed in a cloth bag with 800 mesh, and then is distilled with a steaming furnace to obtain a distillate for later use.

In step (6), the second fermentation broth is distilled to no distillate to obtain distilled fermentation broth for later use; the distilled fermentation broth, the distillate obtained in step (5) and the organic selenium are completely mixed to obtain a second mixed liquid; and olive oil is added into the second mixed liquid with a volume ratio of the second mixed liquid and the olive oil of 1:5-10 to obtain the biological enzyme.

### Embodiment 2

A biological enzyme for killing viruses and a preparation method thereof are provided, and the biological enzyme is made from the following raw materials: 500 g of the Artemisia argyi, 300 g of the Lactuca sativa, 300 g of the Allium tuberosum, 400 g of the Allium cepiforme, 300 g of the Litsea rubescensLeeomte, 150 g of the Glycyrrhiza uralensis, 100 g of the Allium sativum, 150 g of the Mentha canadensis, 200 g of the Allium mongolicum, 150 g of the Lonicera japonica, 120 g of the Sargassum, 30 g of the monazite, 35 g of the calcium carbonate, 20 g of the FOS, 12 g of the IMO, and 40 g of the organic selenium.

The preparation method of the biological enzyme includes the following steps (1)-(6).

In step (1), the Lactuca sativa, the Allium tuberosum, the Litsea rubescensLecomte, the Glycyrrhiza uralensis, the Allium mongolicum and the Lonicera japonica are dried individually according to the weight of the raw materials to obtain dried materials, the dried materials are placed in the grinder for grinding to obtain grinded materials, the grinded materials are decocted with the purified water to obtain decocted materials, and the decocted raw materials are filtered to obtain size; and step (1) further includes the following steps 1 and 2.

In step 1, the Artemisia argyi and the Mentha canadensis are merged to obtain a mixture, the mixture is soaked in water for 1 h to obtain a soaked mixture, the soaked mixture is distilled by using the steam distillation to obtain a distilled mixture, the distilled mixture is extracted for once under reduced pressure by adding water to obtain a first extracted mixture, the first extracted mixture is filtered to obtain a first filtrate, and the first filtrate is concentrated to a first target relative density to obtain a first liquid medicine for later use.

In step 2, the Allium sativum and the Allium cepiforme are pulverized to obtain pulverized mixture, water is added to the pulverized mixture to perform a warm infusion in a closed environment for 3 h to obtain an infused mixture, the ethanol is added into the infused mixture until an alcohol content reaches 50% to obtain a mixture containing alcohol, the mixture containing alcohol is extracted under reduced pressure to obtain a second extracted mixture, the second extracted mixture is filtered to obtain a second filtrate, and the a second filtrate is concentrated to a second target relative density to obtain a second liquid medicine.

In step (2), the first liquid medicine, the second liquid medicine and the size are added into the mixing tank, after stirring evenly, a first mixed liquid is obtained.

In step (3), the first mixed liquid obtained in step (2) is cooked after adding the Sargassum to obtain a cooked mixed liquid, the cooked mixed liquid is filtered to obtain a filtered mixed liquid, and the filtered mixed liquid is cooled to obtain a cooled mixed liquid.

In step (4), 20% of the weight of the FOS and 20% of the weight of the IMO are added into the cooled mixed liquid obtained in step (3), after stirring evenly, an even mixed liquid is obtained, the even mixed liquid is placed in the fermenter to obtain a first fermentation broth; and the first fermentation broth is filtered to obtain a filtered first fermentation broth, and residual FOS, residual IMO and the filtered first fermentation broth placed in the fermenter to obtain a second fermentation broth.

In step (5), the monazite and the calcium carbonate are pulverized to obtain powder; the powder is placed in the furnace at a temperature of 2200° C. to burn for 75 min to obtain burned powder; the burned powder is cooled to obtain cooled powder, the cooled powder is placed in a cloth bag with 1200 mesh, and then is distilled with the steaming furnace to obtain a distillate for later use.

In step (6), the second fermentation broth is distilled to no distillate to obtain distilled fermentation broth for later use; the distilled fermentation broth, the distillate obtained in step (5) and the organic selenium are completely mixed to obtain a second mixed liquid; and olive oil is added into the second mixed liquid with the volume ratio of the second mixed liquid and the olive oil of 1:5-10 to obtain the biological enzyme.

### Comparative Embodiment 1

A biological enzyme for killing viruses and a preparation method thereof are provided, and the biological enzyme is made from the following raw materials: 200 g of the Allium tuberosum, 200 g of the Litsea rubescensLeeomte, 50 g of the Glycyrrhiza uralensis, 50 g of the Allium sativum, 75 g of the Mentha canadensis, 100 g of the Allium mongolicum, 50 g of the Lonicera japonica, 80 g of the Sargassum, 20 g of the monazite, 25 g of the calcium carbonate, 10 g of the FOS, 8 g of the IMO, and 20 g of the organic selenium.

The preparation method of the biological enzyme includes the following steps (1)-(6).

In step (1), the Allium tuberosum, the Litsea rubescensLeeomte, the Glycyrrhiza uralensis, the Allium mongolicum and the Lonicera japonica are dried individually according to the weight of the raw materials to obtain dried materials, the dried materials are placed in the grinder for grinding to obtain grinded materials, the grinded materials are decocted with the purified water to obtain decocted materials, and the decocted materials are filtered to obtain size; and step (1) further includes the following steps 1 and 2.

In step 1, the Mentha canadensis is merged to obtain a mixture, the mixture is distilled by using the steam distillation to obtain a distilled mixture, the distilled mixture is extracted for once under reduced pressure by adding water to obtain a first extracted mixture, the first extracted mixture is filtered to obtain a first filtrate, and the first filtrate is concentrated to a first target relative density to obtain a first liquid medicine for later use.

In step 2, the Allium sativum is pulverized to obtain pulverized Allium sativum, water is added to the pulverized Allium sativum to perform a warm infusion in a closed environment for 3 h to obtain an infused mixture, the ethanol is added into the infused mixture until an alcohol content reaches 50% to obtain a mixture containing alcohol, the mixture containing alcohol is extracted under reduced pressure to obtain a second extracted mixture, the second extracted mixture is filtered to obtain a second filtrate, and the a second filtrate is concentrated to a second target relative density to obtain a second liquid medicine.

In step (2), the first liquid medicine, the second liquid medicine and the size are added into the mixing tank, after stirring evenly, a first mixed liquid is obtained.

In step (3), the first mixed liquid obtained in step (2) is cooked after adding the Sargassum to obtain a cooked mixed liquid, the cooked mixed liquid is filtered to obtain a filtered mixed liquid, and the filtered mixed liquid is cooled to obtain a cooled mixed liquid.

In step (4), 20% of the weight of the FOS and 20% of the weight of the IMO are added into the cooled mixed liquid obtained in step (3), after stirring evenly, an even mixed liquid is obtained, the even mixed liquid is placed in the fermenter to obtain a first fermentation broth; and the first fermentation broth is filtered to obtain a filtered first fermentation broth, and residual FOS, residual IMO and the filtered first fermentation broth are placed in the fermenter to obtain a second fermentation broth.

In step (5), the monazite and the calcium carbonate are pulverized to obtain powder; the powder is placed in the furnace at a temperature of 2000° C. to burn for 105 min to obtain burned powder; the burned powder is cooled to obtain cooled powder, the cooled powder is placed in a cloth bag with 800 mesh, and then is distilled with the steaming furnace to obtain a distillate for later use.

In step (6), the second fermentation broth is distilled to no distillate to obtain distilled fermentation broth for later use; the distilled fermentation broth, the distillate obtained in step (5) and the organic selenium are completely mixed to obtain a second mixed liquid; and olive oil is added into the second mixed liquid with the volume ratio of the second mixed liquid and the olive oil of 1:5-10 to obtain the biological enzyme.

### Comparative Embodiment 2

A biological enzyme for killing viruses and a preparation method thereof are provided, and the biological enzyme is made from the following raw materials: 500 g of the Artemisia argyi, 300 g of the Lactuca sativa, 300 g of the Allium tuberosum, 400 g of the Allium cepiforme, 300 g of the Litsea rubescensLeeomte, 150 g of the Glycyrrhiza uralensis, 100 g of the Allium sativum, 150 g of the Mentha canadensis, 200 g of the Allium mongolicum, 150 g of the Lonicera japonica, 120 g of the Sargassum, 35 g of the calcium carbonate, 20 g of the FOS, 12 g of the IMO, and 40 g of the organic selenium.

The preparation method of the biological enzyme includes the following steps (1)-(6).

In step (1), the Lactuca sativa, the Allium tuberosum, the Litsea rubescensLeeomte, the Glycyrrhiza uralensis, the Allium mongolicum and the Lonicera japonica are dried individually according to the weight of the raw materials to obtain dried materials, the dried materials are placed in the grinder for grinding to obtain grinded materials, the grinded materials are decocted with the purified water to obtain decocted materials, and the decocted materials are filtered to obtain size; and step (1) further includes the following steps 1 and 2.

In step 1, the Artemisia argyi and the Mentha canadensis are merged to obtain a mixture, the mixture is soaked in water for 1 h to obtain a soaked mixture, the soaked mixture is distilled by using the steam distillation to obtain a distilled mixture, the distilled mixture is extracted for once under reduced pressure by adding water to obtain a first extracted mixture, the first extracted mixture is filtered to obtain a first filtrate, and the first filtrate is concentrated to a first target relative density to obtain a first liquid medicine for later use.

In step 2, the Allium cepiforme and the Allium sativum are pulverized to obtain pulverized mixture, water is added to the pulverized mixture to perform a warm infusion in a closed environment for 3 h to obtain an infused mixture, the ethanol is added into the infused mixture until an alcohol content reaches 50% to obtain a mixture containing alcohol, the mixture containing alcohol is extracted under reduced pressure to obtain a second extracted mixture, the second extracted mixture is filtered to obtain a second filtrate, and the a second filtrate is concentrated to a second target relative density to obtain a second liquid medicine.

In step (2), the first liquid medicine, the second liquid medicine and the size are added into the mixing tank, after stirring evenly, a first mixed liquid is obtained.

In step (3), the first mixed liquid obtained in step (2) is cooked after adding the Sargassum to obtain a cooked mixed liquid, the cooked mixed liquid is filtered to obtain a filtered mixed liquid, and the filtered mixed liquid is cooled to obtain a cooled mixed liquid.

In step (4), 20% of the weight of the FOS and 20% of the weight of the IMO are added into the cooled mixed liquid obtained in step (3), after stirring evenly, an even mixed liquid is obtained, the even mixed liquid is placed in the fermenter to obtain a first fermentation broth; and the first fermentation broth is filtered obtain a filtered first fermentation broth, and residual FOS, residual IMO and the filtered first fermentation broth are placed in the fermenter to obtain a second fermentation broth.

In step (5), the calcium carbonate is pulverized to obtain powder; the powder is placed in the furnace at a temperature of 2200° C. to burn for 75 min to obtain burned powder; the burned powder is cooled to obtain cooled powder, the cooled powder is placed in a cloth bag with 1200 mesh, and then is distilled with the steaming furnace to obtain a distillate for later use.

In step (6), the second fermentation broth is distilled to no distillate to obtain distilled fermentation broth for later use; the distilled fermentation broth, the distillate obtained in step (5) and the organic selenium are completely mixed to obtain a second mixed liquid; and olive oil is added into the second mixed liquid with the volume ratio of the second mixed liquid and the olive oil of 1:5-10 to obtain the biological enzyme.

### Experimental Embodiment 1

The above products of the embodiments and the comparative embodiments are entrusted to Wuhan Virus Institute for testing, and a specific test process is as follows: Vero E6 cells are inoculated on a 48-well cell culture plate with a ratio of 1.5×10⁴ cells per well, after incubating the Vero E6 cells in an incubator containing 5% of carbon dioxide (CO₂) at a temperature of 37° C. for 12-16 h, supernatant of cells in the 48-well cell culture plate is removed, and then the cells are incubated for 1 h. Syndrome coronavirus 2 (SARS-COV-2) viruses are added into the 48-well cell culture plate in a biological safety protection third-level (BSL-3) laboratory, a multiplicity of infection (MOI) is 0.05, after incubating for 1 h, supernatant of the cells in the 48-well cell culture plate is removed, the 48-well cell culture plate is washed by phosphate buffered saline (PBS) solution, different concentrations of the products of the embodiments or products of the comparative embodiments are added into the 48-well cell culture plate, 24 h after infection, supernatant in the 48-well cell culture plate is collected. 150 microliters (μL) per well of the supernatant is collected, and added into 270 μL of lysis solution for inactivation, inactivated sample tubes are soaked in a disinfectant, and then taking out of the BSF-3 laboratory.

TABLE 1
Antibacterial effects of sample stock solution of the
embodiment 1 on escherichia coli

| | | | Air virus content | |
|---|---|---|---|---|
| | | | (50% tissue culture | |
| | | | infectious dose | |
| | | Test | abbreviated as | Killing |
| | Action | serial | TCID50, a unit | rate |
| Virus name | time | number | of cubic meter) | (%) |
| Influenza A | 0 (canine | 1 | 3.24 × 10⁶ | |
| (H1N1) virus | kidney | 2 | 2.40 × 10⁶ | |
| Host name: Madin | abbreviated | 3 | 3.24 × 10⁶ | |
| Darby canine | as CK) | | | |
| kidney (MDCK) | 2 h | 1 | <1.62 × 10² | >99 |
| cell | | 2 | <1.62 × 10² | >99 |
| | | 3 | <1.62 × 10² | >99 |

TABLE 2
effects of products in the disclosure on the H1N1 virus

| | | | Virus titer | Average total | Average total | |
|---|---|---|---|---|---|---|
| | Action | | logarithmic | number of | number of | Virus |
| Experimental | concentration | | value | viruses | viruses | killing |
| virus and host | and time | Group | lgTCID50/mL | lgTCID50/mL | 1gTCID50/mL | rate % |
| H1N1 virus | Original | Embodiment 1 | <1.5 | <1.5 | <31.6 | >99.99 |
| Host name: | solution for | Embodiment 2 | <1.5 | | | |
| MDCK cell | 24 hours | Comparative | 4.5 | 4.55 | 2.40 × 10⁵ | |
| | | embodiment 1 | | | | |
| | | Comparative | 4.6 | | | |
| | | embodiment 2 | | | | |

Experimental conclusion are as follows. The products of the disclosure have a killing rate of over 99.99% against the H1N1 virus in the air. After being made into the original solution, the killing rate of the H1N1 virus is greater than 99.99%, which has been verified that the products do indeed have broad-spectrum antiviral effects. Apparently, the above embodiments are merely for the purpose of clearly illustrating the embodiments of the disclosure, and are not a limitation on the embodiments of the disclosure. The purpose is to enable those skilled in the art to understand a content of the disclosure and implement it accordingly, and does not limit the scope of protection of the disclosure. Any modifications, equivalent substitutions, and improvements made within a spirit and principle of the disclosure shall be included within the scope of protection of the claims of the disclosure.

What is claimed is:

1. A biological enzyme for killing viruses, wherein the biological enzyme is made from the following raw materials:
300-500 grams (g) of Artemisia argyi, 200-300 g of Lactuca sativa, 200-300 g of Allium tuberosum, 300-400 g of Allium cepiforme, 200-300 g of Litsea rubescensLeeomte, 50-150 g of Glycyrrhiza uralensis, 50-100 g of Allium sativum, 75-150 g of Mentha canadensis, 100-200 g of Allium mongolicum, 50-150 g of Lonicera japonica, 80-120 g of Sargassum, 20-30 g of monazite, 25-35 g of calcium carbonate, 10-20 g of fructo oligosaccharide (FOS), 8-12 g of isomaltooligosaccharide (IMO), and 20-40 g of organic selenium.

2. The biological enzyme for killing viruses as claimed in claim 1, wherein the biological enzyme is made from the following raw materials: 300 g of the Artemisia argyi, 200 g of the Lactuca sativa, 200 g of the Allium tuberosum, 300 g of the Allium cepiforme, 200 g of the Litsea rubescensLeeomte, 50 g of the Glycyrrhiza uralensis, 50 g of the Allium sativum, 75 g of the Mentha canadensis, 100 g of the Allium mongolicum, 50 g of the Lonicera japonica, 80 g of the Sargassum, 20 g of the monazite, 25 g of the calcium carbonate, 10 g of the FOS, 8 g of the IMO, and 20 g of the organic selenium.

3. The biological enzyme for killing viruses as claimed in claim 1, wherein the biological enzyme is made from the following raw materials: 500 g of the Artemisia argyi, 300 g of the Lactuca sativa, 300 g of the Allium tuberosum, 400 g of the Allium cepiforme, 300 g of the Litsea rubescensLeeomte, 150 g of the Glycyrrhiza uralensis, 100 g of the Allium sativum, 150 g of the Mentha canadensis, 200 g of the Allium mongolicum, 150 g of the Lonicera japonica, 120 g of the Sargassum, 30 g of the monazite, 35 g of the calcium carbonate, 20 g of the FOS, 12 g of the IMO, and 40 g of the organic selenium.

4. The biological enzyme for killing viruses as claimed in claim 1, wherein a preparation method of the biological enzyme for killing viruses comprises:
(1) drying individually, according to the weight of the raw materials, the Lactuca sativa, the Allium tuberosum, the Litsea rubescensLecomte, the Glycyrrhiza uralensis, the Allium mongolicum and the Lonicera japonica to obtain dried materials, placing the dried materials in a grinder for grinding to obtain grinded materials, decocting the grinded materials with purified water to obtain decocted materials, and filtering the decocted materials to obtain size;
merging the Artemisia argyi and the Mentha canadensis to obtain a mixture, soaking the mixture in water for 1 hour (h) to obtain a soaked mixture, distilling the soaked mixture by using a steam distillation to obtain a distilled mixture, extracting the distill mixture for once under reduced pressure by adding water to obtain a first extracted mixture, filtering the first extracted mixture to obtain a first filtrate, and concentrating the first filtrate to a first target relative density to obtain a first liquid medicine for later use;
pulverizing the Allium sativum and the Allium cepiforme to obtain pulverized mixture, adding water to the pulverized mixture to perform a warm infusion in a closed environment for 3 h to obtain an infused mixture, adding ethanol into the infused mixture until an alcohol content of the infused mixture reaches 50% to obtain a mixture containing alcohol, extracting the mixture containing alcohol under reduced pressure to obtain a second extracted mixture, filtering the second extracted mixture to obtain a second filtrate, and concentrating the second filtrate to a second target relative density to obtain a second liquid medicine;
(2) adding the first liquid medicine, the second liquid medicine and the size into a mixing tank and stirring evenly to obtain a first mixed liquid;
(3) cooking the first mixed liquid obtained in step (2) after adding the Sargassum to obtain a cooked mixed liquid, filtering the cooked mixed liquid to obtain a filtered mixed liquid, and cooling the filtered mixed liquid to obtain a cooled mixed liquid;
(4) adding 20% of the weight of the FOS and 20% of the weight of the IMO into the cooled mixed liquid obtained in step (3) and stirring evenly to obtain an even mixed liquid, placing the even mixed liquid in a fermenter to obtain a first fermentation broth; filtering the first fermentation broth to obtain a filtered first fermentation broth, and placing the residual FOS, the residual IMO and the filtered first fermentation broth in the fermenter to obtain a second fermentation broth;
(5) pulverizing the monazite and the calcium carbonate to obtain powder; placing the powder in a furnace at a temperature of 2000-2200 degrees Celsius (° C.) to burn for 75-105 minutes (min) to obtain burned powder; cooling the burned powder to obtain cooled powder, placing the cooled powder in a cloth bag with 800-1200 mesh and then distilling the cooled powder with a steaming furnace to obtain a distillate for later use; and
(6) distilling the second fermentation broth to no distillate to obtain distilled fermentation broth for later use; completely mixing the distilled fermentation broth, the distillate obtained in step (5) and the organic selenium to obtain a second mixed liquid; and adding olive oil into the second mixed liquid with a volume ratio of the second mixed liquid and the olive oil of 1:5-10 to obtain the biological enzyme.

5. The biological enzyme for killing viruses as claimed in claim 1, wherein the biological enzyme is applied to prepare an antiviral product.

6. The biological enzyme for killing viruses as claimed in claim 5, wherein the viruses comprise an influenza virus.

7. The biological enzyme for killing viruses as claimed in claim 5, wherein the viruses comprise an influenza A virus.

8. The biological enzyme for killing viruses as claimed in claim 7, wherein the influenza A virus is a H1N1 virus.

9. The biological enzyme for killing viruses as claimed in claim 1, wherein a weight ratio of the Lactuca sativa: the Allium tuberosum: the Litsea rubescensLeeomte is 1:1: 1, and a weight ratio of the Glycyrrhiza uralensis and the Lonicera japonica is 1:1.

10. A preparation method of a biological enzyme for killing viruses, comprising:
(1) providing 300-500g of Artemisia argyi, 200-300 g of Lactuca sativa, 200-300 g of Allium tuberosum, 300-400 g of Allium cepiforme, 200-300 g of Litsea rubescensLeeomte, 50-150 g of Glycyrrhiza uralensis, 50-100 g of Allium sativum, 75-150 g of Mentha canadensis, 100-200 g of Allium mongolicum, 50-150 g of Lonicera japonica, 80-120 g of Sargassum, 20-30 g of monazite, 25-35 g of calcium carbonate, 10-20 g of FOS, 8-12 g of IMO, and 20-40 g of organic selenium;
(2) drying individually, according to the weight of the raw materials, the Lactuca sativa, the Allium tuberosum, the Litsea rubescensLeeomte, the Glycyrrhiza uralensis, the Allium mongolicum and the Lonicera japonica to obtain dried materials, placing the dried materials in a grinder for grinding to obtain grinded materials, decocting the grinded materials with purified water to obtain decocted materials, and filtering the decocted materials to obtain size;
(3) merging the Artemisia argyi and the Mentha canadensis to obtain a mixture, soaking the mixture in water for 1 h to obtain a soaked mixture, distilling the soaked mixture by using a steam distillation to obtain a distilled mixture, extracting the distill mixture for once under reduced pressure by adding water to obtain a first extracted mixture, filtering the first extracted mixture to obtain a first filtrate, and concentrating the first filtrate to a first target relative density to obtain a first liquid medicine for later use; and
(4) pulverizing the Allium sativum and the Allium cepiforme to obtain pulverized mixture, adding water to the pulverized mixture to perform a warm infusion in a closed environment for 3 h to obtain an infused mixture, adding ethanol into the infused mixture until an alcohol content of the infused mixture reaches 50% to obtain a mixture containing alcohol, extracting the mixture containing alcohol under reduced pressure to obtain a second extracted mixture, filtering the second extracted mixture to obtain a second filtrate, and concentrating the second filtrate to a second target relative density to obtain a second liquid medicine;
(5) adding the first liquid medicine, the second liquid medicine and the size into a mixing tank and stirring evenly to obtain a first mixed liquid;
(6) cooking the first mixed liquid obtained in step (4) after adding the Sargassum to obtain a cooked mixed liquid, filtering the cooked mixed liquid to obtain a filtered mixed liquid, and cooling the filtered mixed liquid to obtain a cooled mixed liquid;
(7) adding 20% of the weight of the FOS and 20% of the weight of the IMO into the cooled mixed liquid obtained in step (5) and stirring evenly to obtain an even mixed liquid, placing the even mixed liquid in a fermenter to obtain a first fermentation broth; filtering the first fermentation broth to obtain a filtered first fermentation broth, and placing the residual FOS, the residual IMO and the filtered first fermentation broth in the fermenter to obtain a second fermentation broth;
(8) pulverizing the monazite and the calcium carbonate to obtain powder; placing the powder in a furnace at a temperature of 2000-2200° C. to burn for 75-105 min to obtain burned powder; cooling the burned powder to obtain cooled powder, placing the cooled powder in a cloth bag with 800-1200 mesh and then distilling the cooled powder with a steaming furnace to obtain a distillate for later use; and
(9) distilling the second fermentation broth to no distillate to obtain distilled fermentation broth for later use; completely mixing the distilled fermentation broth, the distillate obtained in step (7) and the organic selenium to obtain a second mixed liquid; and adding olive oil into the second mixed liquid with a volume ratio of the second mixed liquid and the olive oil of 1:5-10 to obtain the biological enzyme.

11. The preparation method as claimed in claim 10, wherein the biological enzyme is made from the following raw materials: 300 g of the Artemisia argyi, 200 g of the Lactuca sativa, 200 g of the Allium tuberosum, 300 g of the Allium cepiforme, 200 g of the Litsea rubescensLeeomte, 50 g of the Glycyrrhiza uralensis, 50 g of the Allium sativum, 75 g of the Mentha canadensis, 100 g of the Allium mongolicum, 50 g of the Lonicera japonica, 80 g of the Sargassum, 20 g of the monazite, 25 g of the calcium carbonate, 10 g of the FOS, 8 g of the IMO, and 20 g of the organic selenium.

12. The preparation method as claimed in claim 10, wherein the biological enzyme is made from the following raw materials: 500 g of the Artemisia argyi, 300 g of the Lactuca sativa, 300 g of the Allium tuberosum, 400 g of the Allium cepiforme, 300 g of the Litsea rubescensLecomte, 150 g of the Glycyrrhiza uralensis, 100 g of the Allium sativum, 150 g of the Mentha canadensis, 200 g of the Allium mongolicum, 150 g of the Lonicera japonica, 120 g of the Sargassum, 30 g of the monazite, 35 g of the calcium carbonate, 20 g of the FOS, 12 g of the IMO, and 40 g of the organic selenium.

13. The preparation method as claimed in claim 11, wherein step (8) comprises:
pulverizing the monazite and the calcium carbonate to obtain the powder; placing the powder in the furnace at a temperature of 2000° C. to burn for 105 min to obtain the burned powder; cooling the burned powder to obtain the cooled powder, placing the cooled powder in the cloth bag with 800 mesh and distilling the cooled powder with the steaming furnace to obtain the distillate.

14. The preparation method as claimed in claim 12, wherein step (8) comprises:
pulverizing the monazite and the calcium carbonate to obtain the powder; placing the powder in the furnace at the temperature of 2200° C. to burn for 75 min to obtain the burned powder; cooling the burned powder to obtain the cooled powder, placing the cooled powder in the cloth bag with 1200 mesh and distilling the cooled powder with the steaming furnace to obtain the distillate.

15. An application method of the biological enzyme for killing viruses as claimed in claim 1, comprising:
applying the biological enzyme to prepare an antiviral product.

16. The application method of the biological enzyme for killing virus as claimed in claim 15, wherein the viruses comprise an influenza virus.

17. The application method of the biological enzyme for killing virus as claimed in claim 16, wherein the influenza virus is an influenza A virus.

18. The application method of the biological enzyme for killing virus as claimed in claim 17, wherein the influenza A virus is a H1N1 virus.

19. A preparation method of a biological enzyme for killing viruses, comprising:
(1) processing Lactuca sativa, Allium tuberosum, Litsea rubescensLeeomte, Glycyrrhiza uralensis, Allium mongolicum, and Lonicera japonica based on weight of the Lactuca sativa, the Allium tuberosum, the Litsea rubescensLeeomte, the Glycyrrhiza uralensis, the Allium mongolicum and the Lonicera japonica to obtain size; processing Artemisia argyi and Mentha canadensis based on weight of the Artemisia argyi and the Mentha canadensis to obtain a first liquid medicine; and processing Allium sativum and Allium cepiforme based on weight of the Allium sativum and the Allium cepiforme to obtain a second liquid medicine;
(2) adding the first liquid medicine, the second liquid medicine and the size into a mixing tank and stirring evenly to obtain a first mixed liquid;
(3) cooking the first mixed liquid obtained in step (2) after adding the Sargassum to obtain a cooked mixed liquid, filtering the cooked mixed liquid to obtain a filtered mixed liquid, and cooling the filtered mixed liquid to obtain a cooled mixed liquid;
(4) adding 20% of the weight of the FOS and 20% of the weight of the IMO into the cooled mixed liquid obtained in step (3) and stirring evenly to obtain an even mixed liquid, placing the even mixed liquid in a fermenter to obtain a first fermentation broth; filtering the first fermentation broth to obtain a filtered first fermentation broth, and placing the residual FOS, the residual IMO and the filtered first fermentation broth in the fermenter to obtain a second fermentation broth;
(5) pulverizing the monazite and the calcium carbonate to obtain powder; placing the powder in a furnace at a temperature of 2000-2200 degrees Celsius (° C.) to burn for 75-105 minutes (min) to obtain burned powder; cooling the burned powder to obtain cooled powder, placing the cooled powder in a cloth bag with 800-1200 mesh and then distilling the cooled powder with a steaming furnace to obtain a distillate for later use; and
(6) distilling the second fermentation broth to no distillate to obtain distilled fermentation broth for later use; completely mixing the distilled fermentation broth, the distillate obtained in step (5) and the organic selenium to obtain a second mixed liquid; and adding olive oil into the second mixed liquid with a volume ratio of the second mixed liquid and the olive oil of 1:5-10 to obtain the biological enzyme;
wherein the weight of the Artemisia argyi is 300-500 g, the weight of the Lactuca sativa is 200-300 g, the weight of the Allium tuberosum is 200-300 g, the weight of the Allium cepiforme is 300-400 g, the weight of the Litsea rubescensLeeomte is 200-300 g, the weight of the Glycyrrhiza uralensis is 50-150 g, the weight of the Allium sativum is 50-100 g, the weight of the Mentha canadensis is 75-150 g, the weight of the Allium mongolicum is 100-200 g, the weight of the Lonicera japonica is 50-150 g, the weight of the Sargassum is 80-120 g, the weight of the monazite is 20-30 g, the weight of the calcium carbonate is 25-35 g, the weight of the FOS is 10-20 g, the weight of the IMO is 8-12 g, and the weight of the organic selenium is 20-40 g.

20. The preparation method as claimed in claim 19, wherein the processing Lactuca sativa, Allium tuberosum, Litsea rubescensLeeomte, Glycyrrhiza uralensis, Allium mongolicum, and Lonicera japonica based on weight of the Lactuca sativa, the Allium tuberosum, the Litsea rubescensLeeomte, the Glycyrrhiza uralensis, the Allium mongolicum and the Lonicera japonica to obtain size specifically comprises:
drying individually, according to the weight of the raw materials, the Lactuca sativa, the Allium tuberosum, the Litsea rubescensLeeomte, the Glycyrrhiza uralensis, the Allium mongolicum and the Lonicera japonica to obtain dried materials, placing the dried materials in a grinder for grinding to obtain grinded materials, decocting the grinded materials with purified water to obtain decocted materials, and filtering the decocted materials to obtain the size;
wherein the processing Artemisia argyi and Mentha canadensis based on weight of the Artemisia argyi and the Mentha canadensis to obtain a first liquid medicine specifically comprises:
merging the Artemisia argyi and the Mentha canadensis to obtain a mixture, soaking the mixture in water for 1 h to obtain a soaked mixture, distilling the soaked mixture by using a steam distillation to obtain a distilled mixture, extracting the distill mixture for once under reduced pressure by adding water to obtain a first extracted mixture, filtering the first extracted mixture to obtain a first filtrate, and concentrating the first filtrate to a first target relative density to obtain the first liquid medicine; and
wherein the processing Allium sativum and Allium cepiforme based on weight of the Allium sativum and the Allium cepiforme to obtain a second liquid medicine specifically comprises:
pulverizing the Allium sativum and the Allium cepiforme to obtain pulverized mixture, adding water to the pulverized mixture to perform a warm infusion in a closed environment for 3 h to obtain an infused mixture, adding ethanol into the infused mixture until an alcohol content of the infused mixture reaches 50% to obtain a mixture containing alcohol, extracting the mixture containing alcohol under reduced pressure to obtain a second extracted mixture, filtering the second extracted mixture to obtain a second filtrate, and concentrating the second filtrate to a second target relative density to obtain the second liquid medicine.
